(19) United States
(12) Patent Application Publication
(10) Pub. No.: US 2003/0138376 A1
(43) Pub. Date: Jul. 24, 2003

(54) Dosing form for reagents, use of said dosing form in organic chemical synthesis and production of said dosing form

(75) Inventors: Thomas Ruhland, Valby (DK); Per Holm, Vanlose (DK); Kirsten Schultz, Roskilde (DK); Jannie Egeskov Holm, Valby (DK); Kim Andersen, Virum (DK)

Correspondence Address: DARBY & DARBY P.C., 805 Third Avenue, New York, NY, 10022, US

(73) Assignee: H. Lundbeck A/S, Valby-Copenhagen (DK)

(21) Appl. No.: 10/245,836

(22) Filed: Sep. 16, 2002

Related U.S. Application Data
(63) Continuation of application No. PCT/DK01/00185, filed on Mar. 16, 2001.

(30) Foreign Application Priority Data
Mar. 17, 2000 (DK) PA200000451

(51) Int. Cl. A61K 49/00; G01N 33/543

(52) U.S. Cl. 424/9.1; 436/518

(57) ABSTRACT

A dosing form for at least one solid reagent for use in conventional organic and inorganic synthesis, in parallel synthesis, and in split and mix synthesis in combinatorial chemistry is provided as compressed tablets each containing the same predetermined amount of said at least one reagent embedded in a polymer matrix comprising beads of a polymer insoluble in the solvents for the intended synthesis, which tablets are capable of disintegrating in said solvent for release of the at least one reagent and disperse the matrix as polymer beads into the solvent. The polymer beads forming the matrix and the reagents of the dosing form can easily be removed by filtration in order to separate these from a formed soluble product.

In a method for producing the dosing form, beads of one or more polymers are mixed with the reagents and compressed into tablets after pre-treatment with an aprotic organic solvent.

This application is a continuation of International Application No. PCT/DK01/00185, filed Mar. 16, 2001. The prior application is hereby incorporated by reference, in its entirety.

### FIELD OF THE INVENTION

The present invention relates to the dosing of solid reagents in the organic and inorganic chemical field. In particular the invention deals with such dosing forms for use in parallel synthesis or mix and split synthesis in the organic chemical field e.g. combinatorial chemistry and medicinal chemistry.

### BACKGROUND FOR THE INVENTION

Synthetic as well as analytical chemistry involve numerous process steps comprising addition of chemicals especially within parallel synthesis or mix and split synthesis in the organic chemical field e.g. combinatorial chemistry and medicinal chemistry.

Parallel syntheses have become important tools in the search for new compounds in e.g. the pharmaceutical industry and material sciences. Using these concepts, a large number of compounds are synthesized. Parallel synthesis is a particular form for organisation of chemical syntheses where a large number of chemical syntheses simultaneously are performed separately in order to obtain a large number of new single compounds typically for research purposes. For example parallel synthesis can be used to generate a large number, often hundreds or more, of analogues of a particular molecule in order to determine which analogue has the most desirable activity in a specific assay.

Combinatorial chemistry is a form of parallel synthesis where the order and the features of the individual steps are performed using a particular combinatorial approach.

In order to carry out parallel synthesis, a large number of additions, and separations of substances are necessary.

In certain parallel syntheses where a large number of reactions are performed simultaneously, the time consumed by the individual weighing out and distributing the required reagents is considerable. Further errors and mistakes inevitably occur during the required large number of individual weighings.

Additionally, the reagents may be hygroscopic or oxygen sensitive and thus require special measures, especially during weighing, which are additionally time consuming and may confer additionally inaccuracy e.g. due to partially degradation or conversion of the reagents.

Further, contact with the reagents may involve a health risk to the staff performing the syntheses.

Thus there is a need for simple dosing means as alternative to the weighing out and distribution of reagents hitherto used in parallel synthesis and split and mix synthesis in order to reduce the time consumption and increase the through-put of the synthesis; decrease the health risk for the personnel and protect the reagents against the deteriorating effect of oxygen and moisture.

The use of tablets as dosing form for different types of substances is conventional within other technical areas. Thus in the pharmaceutical industries drugs for oral administration are compressed into tablets usually together with various extenders and adjuvants. These tablets as well as tablets produced in other industries, such as detergent tablets, are intended for disintegration and at least partial dissolution usually in an aqueous environment. Generally, these known types of tablets are not suitable as dosing forms in parallel synthesis since they, besides the desired reagents, introduce various adjuvants etc., the presence of which is unacceptable in the synthesis medium and difficult to remove therefrom.

WO 99/04895 discloses dosing forms for solid support polymers comprising capsules, pouches and coated tablets wherein the core of said coated tablets contains a 1:1 mixture of the polymer support and polyethylene glycol. The use of such tablets as a dosing form in parallel synthesis requires a washing step after disintegration of the tablets and prior to chemical reactions in order to remove the polyethylene glycol as well as the coating material.

Atrash et al. (Atrash, B. et al. Angew. Chem. Int. Ed. 2001, 40, No. 5) discloses tablets where the polymer beads are entrapped in an inert polymer matrix which does not disintegrate when suspended in organic solvents.

It has now been found that the above mentioned problems can be solved by a new and inventive process for the manufacture of a dosing form wherein the reagents are embedded in a polymer as tablets with the amount and type of tabletting excipients allowing that the tablets can be used for direct dosage in parallel synthesis without any washing step. In the tablets, the reagents may be embedded in a matrix consisting of polymer beads. When introduced in the synthesis medium, the tablets disintegrate and release the reagents whereas the polymer beads regain their shape and are easily removed by filtration. In certain cases, the polymer can be functionalised with at least one further reagent applied in the reaction.

### SUMMARY OF THE INVENTION

Thus the invention deals with a dosing form for at least one solid reagent for use in chemical synthesis characterized in being compressed tablets. Each tablet containing the same predetermined amount of said at least one reagent embedded in a polymer matrix comprising beads of a polymer insoluble in the solvent for the intended synthesis, which tablets are capable of disintegration in said solvent thereby releasing the at least one reagent and dispersing the matrix as polymer beads into the solvent.

In the syntheses relevant in connection with the present invention, solid reagents embedded in polymers in the shape of beads i.e. particles or small bodies, serve as reagents for reaction with other compounds to obtain a product in solution. After the reaction, the formed product in the solvent have to be separated from the inert insoluble parts of the dosing form and it is an important feature of the invention that this can be done by filtration.

When using a solid reagent embedded in a polymer in form of beads, it is important that the polymer is stable so that it is not degraded to smaller particles or transformed in other respects which would reduce the filterability and thus the advantage of easy separation by filtration.

Further the invention provides a method for production of tablets using conventional tableting equipment.

Surprisingly the tablets can be formed using conventional tabletting equipment without damaging the polymer beads in such a way that the filterability of the resulting dispersion is affected.

In a further preferred embodiment, a pre-treatment of the polymer or the mixture of polymer and reagent and/or additive before tablet compression is provided to improve the flowability, blend uniformity, compressibility and dosing of the material, and therefore reduces the variation in weight, content uniformity and crushing strength of the tablets. Said pre-treatment comprises treatment of the polymer or the mixture of polymer and reagent and/or additive with an aprotic organic solvent.

In still a further embodiment, an addition of a disintegrant (e.g. DM-PEG 2000) increases the ability of disintegration and dispersing of the tablets in a particular solvent.

It is a particular feature of the invention that the formed tablets can be uniformly prepared and are able to disintegrate in a particular solvent to provide a dispersion of the polymer and the at least one reagent in such a way that the reagent is released in total and that the formed dispersion readily can be separated by filtration.

The at least one reagent comprised in the dosing form according to the invention may be any reagent that is useable in organic and/or inorganic chemical synthesis. The reagents should be solid at the temperature for production and storage of the dosing form.

In the present application and in the attached claims, the term “reagent” is used in a broad sense comprising also catalysts such as palladium on carbon.

The at least one reagent may be soluble or non-soluble in the solvent for the intended reaction.

Examples of reagents types for the use in the present invention include: Acetoxylating reagents, acid acceptors, acid catalysts, acrylating reagents, activated ester reagents, activating reagents, acyl anion equivalents, acylating reagents, acylation catalysts, aldolization reagents, alkene addition reagents, alkene metathesis catalysts, alkenylating reagents, alkenylation catalysts, alkoxide bases, alkylating reagents, alkylation catalysts, alkynylating reagents, allenylating reagents, allylating reagents, allylation catalysts, amide bases, amidine bases, aminating reagents, amination catalysts, amine bases, aminoalkylating reagents, aminomethylenating reagents, amphiphilic (electrophilic and nucleophilic) reagents, anion activation reagents, annulation reagents, arene alkylating reagents, arsenating reagents, arylating reagents, arylation catalysts, autoxidation catalysts, azide sources, bases, benzyne precursors, bis-annulating reagents, borylating reagents, bromination reagents, Brønsted-Lowry acids, carbamoylating reagents, carbene precursors, carboalumination reagents, carbon nucleophiles, carbonyl alkenation reagents, carbonylation reagents and catalysts, carboxamininylating reagents, carboxylation reagents, chelation reagents, chiral reagents, cleavage reagents, condensation catalysts, cross-coupling reagents, cuprating reagents, cyanating reagents, cyclization catalysts, cyclization reagents, cycloaddition catalysts, cycloaddition reagents, cyclopropanating reagents, dealkylating reagents, decarboxylating reagents, dehalogenating reagents, dehydrating reagents, dehydrogenating reagents, dehydrohalogenating reagents, deoxygenation reagents, deprotection reagents, derivatization reagents, desilylation reagents, desulfurization reagents, diazoalkane reagents, diazo transfer reagents, dihydroxylation reagents, elimination-inducing reagents, enolate equivalents, enophiles, epoxidizing reagents, ester hydrolis reagents, esterification reagents, fluorinating reagents, fluoroalkylating reagents, formylating reagents, glycosylation reagents, guanylating reagents, halogenating reagents, heteroatom nucleophiles, heterocyclic synthesis reagents, homoenolates, homologating reagents, hydration catalysts, hydride donors, hydroaluminating reagents, hydroborating reagents, hydrocyanation reagents, hydroformylation reagents, hydrogenation catalysts, hydrogen atom donors, hydrogenolysis catalysts, hydrohalogenating reagents, hydrosilylation catalysts and reagents, hydroxyalkylating reagents, hydroxymethylating reagents, isomerization catalysts, ketene precursors, Lewis acids and bases, metalating reagents, methoxylation reagents, methylation reagents, Michael acceptors, Michael addition catalysts, Michael donors, nitrating reagents, nitrosating reagents, nucleotide coupling reagents, oligomerization catalysts, oxidation catalysts, oxidative coupling reagents, oxidizing reagents, oxygenating reagents, peptide coupling reagents, phase-transfer catalysts, reagents, thiophilic reagents, transition metal ligands, trifluoromethylation reagents, vinylating reagents, vinylation catalysts, phenoxylating agents, phosphinylating reagents, phosphitylating reagents, phosphonylating reagents, phosphorylating reagents, photocycloaddition reagents, propargylating reagents, protecting reagents, radical promoters and reagents, rearrangement catalysts, rearrangement reagents, reducing reagents, resolving reagents, ring contraction reagents, ring expansion reagents, selenenylating and selenurating reagents, silylating reagents, stannylating reagents, sulfenylating reagents, sulfinylating reagents, sulfonylating reagents, sulfurating reagents, surfactants, tellurating reagents, thiocyanating reagents, thioetherification reagents, thionating reagents.

Examples of functional classes of reagents and catalysts for the use in the present invention include: Acetals, acids (including inorganic Lewis acids), alcohols and alkoxides, aldehydes, alkenes, alkynes, allenes, aluminum containing reagents, anions (e.g. acetylides and aryl zink halogenides), antimony containing reagents, arsenic reagents, barium containing reagents, bases (organic and inorganic bases), biocatalysts (e.g. yeast, proteines, carbohydrates), bismuth containing reagents, boron reagents (amine complexes, boranes, borates, borohydrides, boronates, boron trifluoride complexes), bromine containing reagents (e.g. bromide ion sources, organic bromine compounds), cadmium containing reagents, calcium containing reagents, carboxylic derivatives (e.g. acid halides, amino acids, ureas, anhydrides, carbonates, carboxylic acids, chloroformates, dicarboxylic acids and esters, esters, hydroxy acids and esters, imides, keto acids, lactams, lactones, nitriles, unsaturated acids and esters), catalysts (organic, inorganic and organometal catalysts), cations (e.g. acylium ions, carbenium ions), cerium containing reagents, cesium containing reagents, chiral reagents (e.g. enolate auxiliaries, ligands), chlorine reagents (including inorganic salts, organochlorine compounds, perchlorates), chromium containing reagents (e.g. oxidizing and non-oxidizing reagents), cobalt containing reagents (inorganic and organocobalt compounds), copper reagents (Cu(I) and Cu(II) compounds), cyclopropanes, dienes and trienes, enzymes, erbium containing reagents, ethers (including epoxides and haloalkyl ethers), europium reagents, Fischer and Schrock carbene complexes, fluorine containing reagents (including fluoride ion sources, hydrofluorinating agents, organofluorine compounds), germanium containing reagents, gold containing reagents, hafnium containing reagents, halonium ions, heterocycles (nitrogen, oxygen, sulfur, and other heteroatoms, including polyheteroatomic heterocycles), hydrides (complex hydrides and inorganic hydrides), hydroxides, indium reagents, iodine containing reagents (including iodide ion sources, iodinating agents, organoiodine compounds), iridium containing reagents, iron containing reagents (including inorganic reagents and organoiron compounds), ketenes and ketene derivatives, ketones (including diketones, halo ketones, keto acids and esters, quinones, α,β-unsaturated ketones), lanthanide containing reagents, lanthanum containing reagents, lead containing reagents, lithium containing reagents (inorganic salts and organolithium compounds), magnesium containing reagents (inorganic salts and organomagnesium compounds), manganese containing reagents (inorganic salts and organomanganese compounds), mercury containing reagents (inorganic salts and organomercury compounds), metal complexing agents (including crown ethers), molybdenum containing reagents (inorganic salts and organomolybdenum compounds), nickel containing reagents (inorganic salts and organonickel compounds), niobium containing reagents, nitrogen containing reagents (including amides, amidines, amines, amino acids and derivatives, ammonium salts, azides, azo compounds, carbamates, cyanamides, cyanides, diazo compounds, diazonium salts, diimides, disilazides, enamines, guanidines, heterocycles, hydrazides, hydrazines and hydrazones, hydroxamic acids, hydroxylamines, imidates, imides, imines, iminium salts, isocyanates, isocyanides, metal amides, nitrates, nitrile oxides, nitriles, nitrites, nitro compounds, nitrones and nitronates, nitroso compounds, nitroxides, oximes, quaternary ammonium salts, ureas, ynamines), orthoesters, osmium containing reagents, oxonium ions, oxygen containing reagents (including heterocycles), palladium containing reagents, peroxides, phenols, phosphorus reagents (including Horner-Wadsworth-Emmons reagents, Homer-Wittig reagents, phosphines and phosphine oxides, phosphinic acid derivatives, phosphinous acid derivatives, phosphonic acid derivatives, phosphonium salts, phosphoranes, phosphoric acid derivatives, phosphorous acid derivatives), platinum containing reagents, potassium containing reagents (inorganic salts and organopotassium compounds), quinones, rhenium containing reagents, rhodium containing reagents, ruthenium containing reagents, samarium containing reagents, selenium containing reagents (including diselenides, electrophilic selenylating reagents, nucleophilic selenenylating reagents, selenocyanates), silicon containing reagents (including alkenylsilanes, alkynylsilanes, enol silanes, metalated silanes, silanes, silazanes, siloxanes and analogs, siloxy compounds, silyl alkanesulfonates, silyl halides), silver containing reagents, sodium reagents (inorganic salts and organosodium compounds), sulfur reagents (including disulfides, electrophilic thiolating reagents, haloalkyl, heterocycles, nucleophilic thiolating reagents, sulfamides, sulfates, sulfenyl halides, sulfides and metalated sulfides, sulfilimines, sulfinates, sulfites, sulfonamides, sulfonates, sulfones and metalated sulfones, sulfonic acids and anhydrides, sulfonium salts, sulfonyl azides, sulfonyl cyanides, sulfonyl halides, sulfonyl hydrazides, sulfonyl isocyanates, sulfoxides and metalated sulfoxides, sulfoximines, sulfuranes, sulfurating reagents, sulfur ylides, thiazolium salts, thioacetals, thioacids and derivatives, thioacylating agents, thiocyanates and isothiocyanates, thiolates, thiols), tantalum containing reagents, tellurium containing reagents, thallium containing reagents, tin containing reagents (including distannanes, halides, inorganic compounds, metalated stannanes, oxides, stannanes, sulfides and selenides, unsaturated tin compounds), titanium containing reagents (inorganic and organo titanium compounds), tungsten containing reagents, uranium containing reagents, vanadium containing reagents (including inorganic salts and vanadium organo compounds), xenon containing reagents, ylides (antimony, arsenic, phosphorus and sulfur ylides), ytterbium containing reagents, zinc containing reagents (inorganic salts and organozinc reagents), zirconium reagents (inorganic and organozirconium reagents).

The polymer for use in dosing forms according to this invention may be any polymer that is insoluble in the relevant solvents, inert to the reaction conditions, capable of being compressed, with or without suitable adjuvants, to form tablets capable of disintegrating in said relevant solvents, and able to reshape as beads after the disintegration of the tablet.

A preferred polymer according to the invention is polystyrene or a functionalized polymer based on polystyrene or of another backbone. Based on polystyrene means that the polymer contains a polystyrene backbone that may be substituted or it may be a copolymer comprising styrene or substituted styrene monomers. The polymer may be a linear polymer or a polymer cross-linked with a cross-linking agent as will be known within the art. An example of a suitable cross-linking agent is divinyl benzene (DVB).

Further preferred polymers in the dosing forms according to the invention are functionalized polystyrene based resins such as polystyrene cross-linked with divinyl benzene (DVB), including polyethylene glycol grafted resins such as the Tentagel® and Argogel® resins, linear polystyrene, polystyrene resins cross-linked with polyethylene glycol including the POEPS (Renil and Meldal, Tetrahedron Letters 37, 6185-88, 1996), and POEPS-3 resins (Buchardt and Meldal, Tetrahedron Letters 39, 8695-8698, 1998), polystyrene resins crosslinked with polyoxybutylene such as the poly(styrene-tetrahydrofuran) resins (JandaGel®) (Toy, P. M.; Janda U. D. Tetrahedron. Lett. 1999, 40, 6329-32), polyoxyethylene polyoxy propylene (POEPOP) resins (Renil and Meldal, supra).

In a further preferred embodiment, the polymers are co-polymerized together with additives to achieve special properties of the beads such as magnetic properties by addition of magnetites or magnetites captured in highly cross-linked polystyrene particles (Scholeiki, I., Perez, J. M. Tetrahedron Lett. 1999, 40:3531-3534 and Prof. Mark Bradley, Dep. of Chemistry, University of Southampton, Presentation at the Conference “High-throughput Synthesis”, Feb. 9-11, 2000).

In another embodiment of the invention at least one of the catalysts or reagents comprised in the dosing form is chemically bonded to the polymer. A number of such polymers containing reactants are listed by Ley et al. (Ley, S. V. et al; J.Chem. Soc., Perkin Trans. 1, 2000, 3815-4195).

In a preferred embodiment of the invention, the dosing form comprises a phosphine and an azo compound of the formula

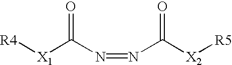

wherein X1 and X2 independently are N or O, and R4 and R5 independently are selected from the group comprising lower alkyl and polymer-bonded equivalents thereof. The phosphine is preferably of the formula R¹R²R³P wherein R¹, R² and R³ independently are selected from the group comprising phenyl, heteroaryl, lower alkyl, phenyl-lower alkyl, heteroaryl-lower aryl and polymer-bonded equivalents thereof. Preferably, one of these reagents is bonded to the polymer. The reagent bonded to the polymer may be either the phosphine or azodicarboxylate.

Dosing forms of this type may be useful in reactions wherein acidic heteroatoms are to be alkylated, e.g. the Mitsunobu reaction, which is an alkylation reaction well-known to those skilled in the art. The use of solid support linked phosphines in the Mitsunobu reaction is described inter alia in (Pelletier and Kincaid, Tetrahedron Letters 41 (2000) 797-800).

In another preferred embodiment of the invention, the dosing form comprises a phosphine and carbon tetrabromide. Preferably, the phosphine is bonded to the polymer. The phosphine is preferably of the formula R¹R²R³P wherein R¹, R² and R³ are as defined above.

Dosing forms of this type may be usefull in reactions wherein basic heteroatoms are to be acylated.

As used herein, the term ‘lower alkyl’ means any branched or unbranched C₁₋₆ alky.

As used herein, the term ‘heteroaryl’ means any heteroaryl selected from the group comprising 2-pyridyl, 3-pyridyl, 4-pyridyl.

As used herein, the term ‘polymer-bonded equivalents’ means any equivalent compound which is chemically bonded to the polymer support through one of the R-groups.

As used herein, the term ‘acidic heteroatom’ means any heteroatom Y in a group —Y—H which is capable of dissociating the proton, and wherein Y is selected from the group comprising N, O, S.

As used herein, the term ‘basic heteroatom’ means any heteroatom Z which is capable of being protonated, and wherein Y is selected from the group comprising N, O, P, S.

As used herein, the term ‘solid reagent’ means any reagent which is solid at the temperature at which the tablets are manufactured including polymer-bonded reactants as well as reactants that are not bonded to polymers.

In one embodiment, the polymer is composed of a mixture of two or more polymers. Mixtures of polymers may be used in the dosing form in order to obtain tablets with more desired properties.

Particular a disintegrating agent may be included to enhance the disintegration of the resulting tablets in a particular solvent. In principle, all disintegrating agents that are inert under the conditions of the intended reaction may be applied. A preferred disintegrating agent is dimethylated polyethylene glycol (DM-PEG), preferably DM-PEG with a molecular weight of about 2000 Da (DM-PEG 2000). Preferably, the amount of polyethylene glycol (PEG) does not exceed 20% by weight of the tablet, more preferred it does not exceed 10% by weight of the tablet, suitably the amount of PEG in the tablets is zero. Preferably, the amount of other tabletting additives as well does not exceed 20% by weight of the tablet, more preferred it does not exceed 10% by weight of the tablet, suitably the amount of other tabletting additives in the tablets is zero.

The choice of polymer or mixture of polymers used may be selected to enhance the disintegration in the solvent of the reaction for which the dosing forms are intended. The polymer composition may for instance be selected to obtain disintegration in protic organic solvents such as methanol or ethanol.

Other additives known within the tabletting area may be used provided that they are chemically inert and insoluble or otherwise acceptable in the reaction medium for which the tablets are intended, for example may silicon(IV)oxide be added, e.g. to avoid problems caused by static electricity.

Generally polymers are marketed as a particulate material where the particles may have different shapes and forms depending on the manufacturing of the polymer. According to the present invention, the polymer is used in form of beads which means small bodies, particles or pellets, where the surfaces are essential smooth and convex and the longest dimension is not larger than 3 fold of the shortest dimension. The forms of the beads may for example be spherical, drop-shaped and ellipsoid.

The size of the polymer beads used according to the invention is selected to enable good filterability which is promoted by large particles, balanced with a desire for a reasonably high specific surface area, which is promoted by small particles. The particle size of the polymer beads is according to the invention selected in the range 20-600 mesh, preferably 100-400 mesh.

The formation of the tablets may be performed in an inert atmosphere in order to prevent deterioration of the reagents due to oxidation by oxygen or absorption of moisture from the atmosphere. As an inert atmosphere, any inert gas may be used as it will be known within the area. Examples of gases for the inert atmosphere are nitrogen and argon.

Tablet formation can be done using conventional tabletting techniques. A mixture containing the reagent(s) and the polymer(s) is formed into tablets by application of a certain mechanical force, possibly after granulation, using a tabletting machine as it will be known within the art.

Tablets may be formed containing various amounts of the polymer support for example in amounts in the range of 5-5000 mg. The ratio of reagent(s) to polymer is selected with due regard to the intended use of the tablets and the mechanical stability of the tablets. Generally at least 50% polymer is needed, preferably 50-90% and more preferably 60-75% polymer based on the total weight of the tablets.

The tablets may be compressed to a desired form and size for example to fit in a device such as a tablet dispenser.

The tablets must have a sufficiently high stability to avoid breaking during package, transportation and dispensing. The crushing strength is a measure for the mechanical stability of tablets. The crushing strength of the tablets must be higher than 5 N, preferably higher than 10 N, in order to have a satisfactory mechanical stability.

It has turned out that a pre-treatment of some or all the ingredients of the dosing form may improve the quality of the resulting dosing forms. Basically the ingredients are pre-treated with an aprotic organic solvent.

The pre-treatment may be performed in different ways depending on the solubility of the reagents in the chosen solvent. If all the reagents are insoluble or almost insoluble in the solvent for the pre-treatment it is made by mixing the polymer or the mixture of the polymer and the reagent and/or additives in the solvent. When a homogenous mixture is obtained, the polymer or the mixture of the polymer and the reagent and/or additives is filtered off and dried whereafter it is ready for tablet formation.

If at least one of the reagents are soluble in the solvent for the pre-treatment, the insoluble part of the ingredients are added to a solution of the soluble part of the ingredients in said solvent. After a homogenous mixture is obtained, the solvent is removed by evaporation.

Pre-treating the powder/powder mixture before tablet formation significantly improves the flowability, blend uniformity, compressibility and dosing of the material, which again improves the uniformity in respect of dose, disintegration time and mechanical stability of the tablet.

The solvent for the pre-treatment may be any aprotic organic solvent. Preferred solvents for use in the pre-treatment are methylene chloride and tetrahydrofuran.

One possible explanation, which should not be construed as limiting the scope of the patent, of the effect of treating the polymer or mixture of polymer and reagents and/or additives with an aprotic solvent is that the surface of the polymer beads are partially swollen by the aprotic organic solvent resulting in agglomeration of the polymer beads with the effect that the treated polymer powder may be compressed into tablets with improved mechanical properties.

The dosing forms according to this invention may be composed to be useable in any protic or aprotic solvent that is suitable for the intended synthesis. The solvent may even be a reagent in the intended reaction for instance if methanol is the solvent in a methoxylation reaction or if a mixture of THF and methylene diiodide is the solvent for a tablet containing polystyrene and samarium metal powder to generate samarium diiodide (Molander, G. A., Alonso-Alija, C. Tetrahedron, 53, 1997, 8067-8084.).

Organic solvents are preferred. Examples of organic solvents that are suitable according to the invention are: methylene chloride, tetrahydrofuran, toluene, acetonitrile, ethylacetate, DMSO, DMF and hexane. Methylene chloride and tetrahydrofuran are preferred solvents.

That a tablet is capable of disintegration in a solvent means that the tablet with application of a minimal mechanical force such as by vortex mixing can disintegrate in the solvent within 30 minutes, preferably within 10 minutes, more preferred within 5 minutes to form a uniform dispersion.

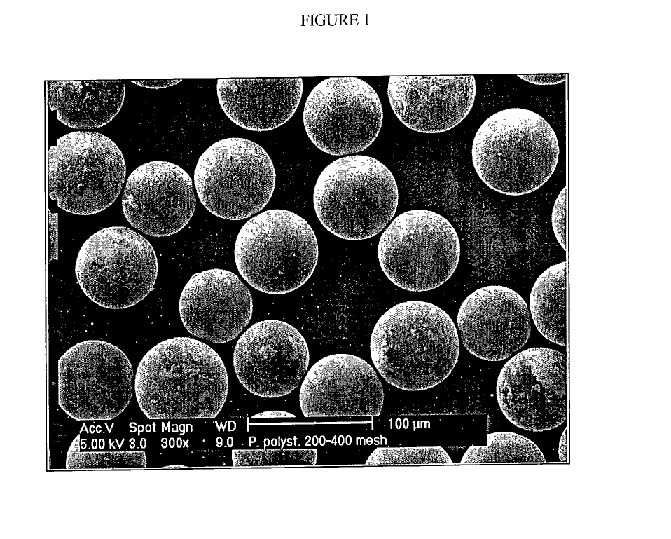

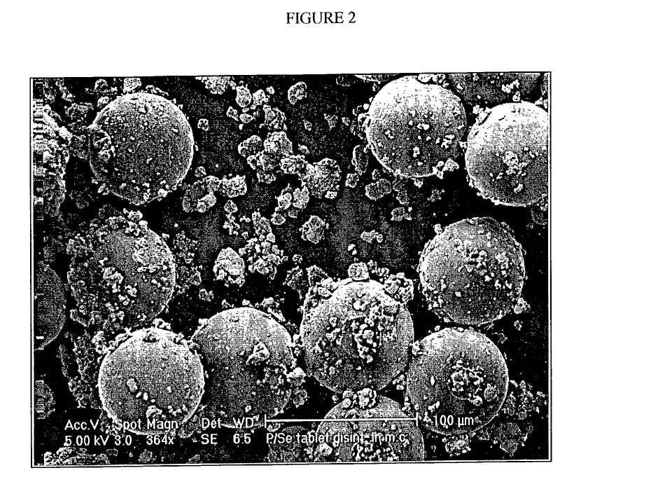

The term “capable of reshaping after the disintegration” means that the polymer beads regain essentially their original shape after the disintegration of a tablet comprising said beads. Further it means that the beads are not mechanical damaged by the tablet compression and subsequent disintegration. Reshaping of the beads can conveniently be evaluated by comparation of SEM pictures of the beads before tablet formation and after disintegration. If the beads are capable of reshaping, the shapes of the beads are not substantially altered and the number of cracks and faults in the beads after the dispersion is not substantially higher than before the tablet formation, cf. FIG. 1 and FIG. 2 for further details.

### BRIEF DESCRIPTION OF FIG. 1 AND FIG. 2

The drawing illustrate an experiment where a polymer and a reagent were compressed into tablets and subsequently disintegrated in a organic solvent.

FIG. 1: SEM of polystyrene beads, 200-400 mesh, before tablet compression.

FIG. 2: SEM of a powder mixture originating from the disintegration of a tablet comprising polystyrene and powder of Selenium (CC-11 in table 1).

For Scanning Electron Microscope (SEM) pictures, the samples were sputter coated with gold/palladium and SEM analysis was performed using a Philips electron microscope XL30.

In FIG. 1, the polystyrene beads can be seen as separate uniform spherical bodies in a narrow range of sizes.

In FIG. 2, polystyrene beads are seen as uniform spherical bodies and the particulate Se powder as smaller particles of irregular non-uniform shapes. The observed beads are all intact without noticeable cracks or damages.

The complete disintegration of the beads as essentially single beads secures that reagent is released in total.

The dosing forms according to the invention are stable and reliable dosing forms that can easily, safely and reliably be distributed to a large number of individual reactions in any form of parallel synthesis and thus increase the throughput of parallel synthesis in a reliable and accurate way.

The invention is now illustrated by specific examples which are presented for illustratory purposes alone and should not be construed as any limitation of the invention.

### EXAMPLES

General Procedures

All reactions were carried out under positive pressure of nitrogen. Unless otherwise noted, starting materials were obtained from commercial suppliers and used without further purification. Tetrahydrofuran (THF) was distilled under N₂ from sodium/benzophenone immediately prior to use. Thin layer chromatography (TLC) was performed on Merck 60 F₂₅₄ 0.25 μm silica gel plates. ¹H NMR and ¹H-decoupled ¹³C NMR spectra were recorded at 500.13 MHz and 125.67 MHz, respectively, on a Bruker Avance DRX 500 instrument. Unless otherwise noted, compounds were measured in deuterated chloroform (99.8%). Chemical shifts for ¹H NMR are reported in ppm with TMS as internal reference. Chemical shifts for 13C NMR are reported in ppm relative to chemical shifts of deuterated solvents. Coupling constants (J values) are in Hertz. The following abbreviations are used for multiplicity of NMR signals: s=singlet, d=doublet, t=triplet, q=quartet, qui=quintet, dd=double doublet and m=multiplet. LC-MS data were obtained on a PE Sciex API150EX equipped with a Heated Nebulizer source operating at 425° C. The LC pumps were Shimadzu 8A series running with a Waters C-18 4.6×50 mm, 3.5 μm column. Solvent A 100% water+0.05% trifluoroacetic acid, solvent B 95% acetonitrile 5% water+0.035% trifluoroacetic acid. Gradient (2 ml/min): 10% B-100% B in 4 min, 10% B for 1 min. Total time including equilibration, 5 min. Injection volume 10 μL from a Gilson 215 Liquid Handler. GC-MS data were obtained on a Varian CP-3800/Saturn 2000 instrument. The column was Varian CP-Sil8 CB-MS Rapid-MS (10×0.53 mm) with He-flow 1.1 mL/min. Temperature gradient was 60° C. to 300° C. in 15 min. The mass detector was operated in EI mode. The compression of tablets was performed at a Korsch EK0 single punch machine. The crushing strength was measured at a Schleuniger 6D tablet hardness tester. Disintegration times of tablets were measured in a glass tube (16×100 mm) with 2 mL solvent by vortex mixing at a speed of approximately 500 Hz with an IKA shaker (KS 125 basic). The process of tablet disintegration was monitored visually and tablets were deemed to be fully disintegrated when a dispersion was formed and no more lumps were present. For Scanning Electron Microscope (SEM) pictures, the resin samples were sputter coated in a Microtech, Polaron SC 7640 using a gold/palladium electrode and SEM analysis was performed using a Philips electron microscope XL30. High resolution mass spectra (HR-MS) were performed at the University of Odense, Department of Chemistry (Odense, Den.) with the peak-matching method using a Varian MAT 311A mass spectrometer. Elemental analyses were performed at the University of Vienna, Department of Physical chemistry (Vienna, Austria), with a Perkin-Elmer 2.400 CHN elemental analyser.

Dimethylated polyethylene glycol (DM-PEG; molecular weight app. 2000 Da) was purchased from Clariant GmbH (Gendorf, Germany) (Material was grinded in a laboratory blender and sieved, particle size varies). Polystyrene resin was purchased from Rapp Polymere GmbH (Tübingen, Germany) (H 1000, 100-200 mesh, cross-linked with 1% divinylbenzene). Diphenylphosphanyl polystyrene was purchased from Senn Chemicals (Dielsdorf, Switzerland) (Cat. No 40258; 1.69 mmol/g; 100-200 mesh, cross-linked with 1% divinylbenzene. Isocyanato methyl polystyrene (approximately 1 mmol/g; 100-200 mesh respectively 200-400 mesh; cross-linked with 1% divinylbenzene) was analogously prepared to Booth, R. J.; Hodges, J. C. J. Am. Chem. Soc 1997, 119: 4882-4886, starting from aminomethyl polystyrene.

Preparation of Tablets

Agglomeration of Polymer Beads

Polystyrene

Polystyrene (25.0 g) was suspended in methylene chloride (150 mL). The polystyrene was filtered through a D3-frite by gravity and dried on the frite at room temperature in vacuo.

The following agglomerates were prepared according to the procedure described above: Diphenylphosphanyl polystyrene, isocyanato methyl polystyrene.

Agglomeration of Mixtures of Polystyrene Beads and Solid Reagents

Samarium/Polystyrene

Polystyrene (10.0 g) was suspended in methylene chloride (60 mL) at room temperature. Samarium powder (3.0 g, approximately 325 mesh; Alfa(®) was added. The suspension was filtered under continuous stirring on a D3-frite by gravity and dried on the frite at room temperature in vacuo.

The following agglomerates were prepared according to the procedure described above: Tin(II) chloride dihydrate/polystyrene (1.00:3.00); Palladium on charcoal/polystyrene (1.00:2.85); Dimethylated polyethylene glycol/polystyrene (1.00:9.00); Potassium carbonate/polystyrene (1.00:2.03); sodium periodate/polystyrene (1.00:2.03); Selenium/polystyrene (1.00:2.85); Aluminium/polystyrene (1.00:4.00); Indium/polystyrene (1.00:4.00); Phenylhydrazine hydrochloride/polystyrene (1.00:2.85)

Agglomeration of Mixtures of Polystyrene Beads and Soluble Reagents

Tetrakis(triphenylphosphine)palladium(0)/polystyrene

Polystyrene (10.0 g) was suspended at room temperature under inert gas in a solution of tetrakis(triphenylphosphine)palladium(0) (1.6 g) in methylene chloride (100 mL). After 15 min, the solvent was slowly and carefully evaporated on a rotavap in vacuo (35° C.).

The following agglomerates were prepared according to the procedure described above: Tetrabromo methane/polystyrene (1.00:2.87); Di-tert-butyl azodicarboxylate/polystyrene (1.00:3.00).

Tablet Compression

The dried agglomerated material was gently crushed by mortar and pistil and screened through a screen size of 710 μm and transferred to the filling device of the single punch tabletting machine. For the tablets containing both functionalised polystyrene beads and a mixture of polystyrene beads and a soluble or insoluble reagent, the mixtures were agglomerated separately and mixed before the screening. The tabletting was performed either manually (10-20 tablets) or automatically with a tabletting speed of 50-90 tablets per hour referred to as up-scaling. The compression force was controlled at a value resulting in tablets having a crushing strength of 8-25 N. Tablets with weights in a range of 80-250 mg were produced. The punch diameters used were in the range of 4- 8 mm with compound cup shape.

Using the described general procedure, tablets with compositions and crushing strength as shown in Table 1 were produced.

TABLE 1
Tablet Properties

| | | Proportion | Agglomer- | Tablet | Tablet | Crushing |
|---|---|---|---|---|---|---|
| | Embedded | of PS^{a)} | ation | weight | diameter | strength |
| Code | Material | [%] | Method | [mg] | [mm] | [N] |
| CC-1 | SnCl₂*2H₂O | 75 | A | 200 | 8 | app.18 |
| CC-2 | Pd/C^{b)} | 77 | B | 250 | 8 | 15 |
| CC-3 | Pd(PPh₃)₄ | 86 | B | 100 | 6 | 15 |
| CC-4 | Pd(dba)₂/P(t-Bu)₃^{c)} | 87 | B | 100 | 6 | not |
| | | | | | | measured |
| CC-5 | Ph-NHNH₂*HCl | 74 | B | 150 | 8 | app. 20 |
| CC-6 | DM-PEG 2000^{d)} | 90 | A | 100 | 6 | 18 |
| CC-7 | K₂CO₃ | 67 | B | 100 | 6 | app. 15 |
| CC-8 | NaIO₄ | 67 | B | 100 | 6 | varies |
| CC-9 | Sm^{e)} | 67 | A | 100 | 6 | app. 15 |
| CC-10 | Sm^{e)} | 77 | B | 100 | 6 | app. 15 |
| CC-11 | Se^{f)} | 77 | B | 100 | 6 | app. 15 |
| CC-12 | Al^{g)} | 80 | B | 100 | 6 | 15 |
| CC-13 | In^{h)} | 80 | B | 100 | 6 | 15 |

^{a)}PS = Polystyrene, 1% divinylbenzene (DVB), 200-400 mesh, Rapp Polymere (Tuebingen, Germany); cat.-No: H 400 00
^{b)}Pd on activated carbon; proportions: Pd (4.8%), H₂O (57.8%); C (37.4%), JMC Johson Matthey, UK
^{c)}Ratio: Pd(dba)₂/P(t-Bu)₃ = 3.59:1.00, dba = dibenzylideneacetone, P(t-Bu)₃ = Tri-tert-butylphosphine (ALPHA)
^{d)}DM-PEG 2000 = dimethylated polyethylene glycol; molecular weight of app. 2000 Da; Clariant GmbH (Gendorf, Germany) material grinded in a laboratory blender, particle size varies
^{e)}Sm-powder; app. 40 mesh, Avocado
^{f)}Se-powder, app. 100 mesh, Aldrich
^{g)}Al-powder (bronze); E. Merck, Darmstadt (Germany)
^{h)}In-powder, app. 325 mesh, ALPHA
Agglomeration method
A) Polystyrene pre-treated with methylene chloride, embedded materials not pre-treated
B) Mixture of Polystyrene and embedded material pre-treated with methylene chloride
Evaluation of tablets

Disintegration of the Tablets

The tablet was placed in a glass tube (16×100 mm) and treated with 2 mL solvent (Table 2). The mixture was agitated by vortex mixing at a speed of approximately 500 Hz with an IKA shaker (KS 125 basic). The progress of tablet disintegration was monitored visually. Tablets were deemed to be fully disintegrated when a dispersion was formed in the tube and no more lumps were present. The results are summarised in Table 2.

TABLE 2
Disintegration of tablets in diferent solvents

| | CH₂Cl₂ | THF | DMF | Toluene | CH₃CN | DMSO | Ethanol |
|---|---|---|---|---|---|---|---|
| Code | (sec) | (min) | (min) | (min) | (min) | (hours) | (hours) |
| CC-1 | <60 | <4.0 | <17.0 | <6 0 | <150 | >24* | >24* |
| CC-2 | <10 | <1.0 | <1.0 | <1 0 | <15.0 | <0.06 | <3 |
| CC-3 | <10 | <2.0 | <1 0 | <4 0 | <3.0 | <12 | >24* |
| CC-4 | <20 | <4.0 | <9.0 | <7.0 | nt | nt | nt |
| CC-5 | <20 | <1.0 | <1.0 | <1.0 | <150 | <12 | >24* |
| CC-6 | <180 | <7.0 | <10.0 | <2.0 | <20.0 | <0.33 | <2 |
| CC-7 | <60 | <4.0 | <1.0 | <1.0 | <20.0 | >24* | >24* |
| CC-8 | <20 | <5.0 | <5.0 | <4.0 | <80 0 | >24* | >24* |
| CC-9 | <90 | <1.0 | <1.0 | <1.0 | <25.0 | >24* | >24* |
| CC-10 | <90 | <1.0 | <1.0 | <1.0 | <420 | >24* | >24* |
| CC-11 | <30 | <1.0 | <1.0 | <1.0 | <420 | >24* | >24* |
| CC-12 | <30 | <2.0 | <1.0 | <1.0 | <35.0 | >24* | >24* |
| CC-13 | <240 | <1.0 | <11.0 | nt | nt | nt | nt |

nt not tested
* not disintegrated within 1 day

Filterability

After disintegration of the tablets, the filterability of the formed dispersion was evaluated by using different filter types. All tablets had formed dispersion that readily could be filtered.

Mechanical Stability of the Polymer

For the analysis of the mechanical stability of the polymer beads, tablets of a mixture of polystyrene and selenium powder were formed. One tablet was added to 2 mL methylene chloride and left until the tablet was fully disintegrated.

A sample of the polymer before tablet formation and a sample of a disintegrated tablet were subjected to SEM analysis using a Philips electron microscope XL30.

The SEM of the polymer before tablet formation shows that the polymer particles are smooth round beads without visible cracks or faults (See FIG. 1).

The SEM of the polymer after disintegration of the tablet shows that the beads are smooth and round without visible deformations and cracks. Further it can be seen that the selenium powder is present as particles between the polymer beads and as such released in total.

This analysis shows that the polymer beads are capable of reshaping after disintegration of the tablet and that no mechanical damage is observed.

Evaluation of Chemical Performance of Embedded Reagents After Agglomeration with Polystyrene and Compression to Tablets

### Example 1

Mitsunobu Reaction by Use of Tablets Comprising Polystyrene, di-tert-butyl Azodicarboxylate and Diphenylphosphanyl Polystyrene in Combination with Tablets of Isocyanato Methyl Polystyrene

The results of the evaluation are summarised in Table 3. A detailed description of the procedure follows:

2-(2-Phenylsulfanyl-ethyl)-2H-naphtho[1,8-cd]isothiazole 1,1-dioxide (entry 1). Two tablets containing in total 0.22 mmol di-tert-butyl azodicarboxylate and 0.29 mmol resin bound diphenylphosphine were added at room temperature to a solution of 2H-naphtho[1,8-cd]isothiazole 1,1-dioxide (21.0 mg, 0.10 mmol) and 2-phenylsulfanyl-ethanol (29.9 mg, 0.20 mmol) in THF (3 mL). After stirring for 16 h, THF (2 mL) and one tablet containing isocyanatomethyl polystyrene (150 mg, 0.15 mmol) was added. The mixture was stirred for 2 h at 60° C. The resin was filtered and washed with methylene chloride (1×1 mL), methanol (1×1 mL) and methylene chloride (1×2 mL). Trifluoroacetic acid (0.4 mL) was added to the combined filtrates and the mixture was stirred for 1.5 h. After evaporation of the solvents in vacuo, the residue was purified by solid phase extraction over silica gel (heptane/ethyl acetate=5:1) to furnish 31.5 mg (89%) of the desired product as a solid (LC-MS: 98% UV-purity and 99% ELSD-purity). An analytical sample was obtained as slightly yellow needles by recrystallization from diethylether (mp: 94° C.). ¹H-NMR δ3.93 (t, 2H, J=8.0), 4.03 (t, 2H, J=7.8), 6.53 (d, 1H, J=6.6), 7.27 (t, 1H, J=6.6), 7.36 (t, 2H, J=7.8), 7.47 (m, 4H), 7.74 (t, 1H, J=7.8), 7.94 (d, 1H, J=7.1), 8.05 (d, 1H, J=8.0).

The following compounds were prepared according to the procedure described above. The treatment with trifluoroacetic acid was omitted in synthesis for compounds of entry 5 and 6.

4-(2-Phenylsulfanylethoxy)-biphenyl (entry 2) was prepared from 2-phenylsulfanyl-ethanol (0.20 mmol) and biphenyl acetic acid. (0.10 mmol). Purification by solid phase extraction (heptane/ethyl acetate=5:1) furnished 27.9 mg (91%) of the desired product as a solid (LC-MS: 89% UV-purity and 87% ELSD-purity). An analytical sample was obtained as colourless needles by recrystallization from diethylether (mp: 100° C.). ¹H-NMR δ3.31 (t, 2H, J=7.1), 4.18 (t, 2H, J=7.1), 6.91 (d, 2H, J=8.5), 7.22 (t, 1H, J=7.5), 7.29-7.32 (m, 3H), 7.39-7.43 (m, 4H), 7.49 (d, 2H, J=8.9), 7.52 (d, 2H, J=8.0).

5-Nitro-2-(2-phenylsulfanylethoxy)-isoindole-1,3-dione (entry 3) was prepared from 2-phenylsulfanyl-ethanol (0.20 mmol) and 5-nitro-isoindole-1,3-dione (0.10 mmol). Purification by solid phase extraction (heptane/ethyl acetate=5:1) furnished 33.0 mg (100%) of the desired product as a solid (LC-MS: 85% UV-purity and 80% ELSD-purity). An analytical sample was obtained as intensively yellow needles by recrystallization from diethylether (mp: 113° C.). ¹H-NMR δ3.27 (t, 2H, J=6.8), 3.99 (t, 2H, J=6.8), 7.09 (t, 1H, J=7.3), 7.21 (t, 2H, J=7.8), 7.38 (d, 1H, J=7.1), 7.98 (d, 2H, J=7.1), 8.58 (dd, 1H, J₁=8.3 and J₁=2.1), 8.60 (d, 1H, J=1.9).

2-Phenylsulfanyl-ethyl)-5-nitro-naphthalene-1-carboxylic acid (entry 4) was prepared from 2-phenylsulfanyl-ethanol (0.20 mmol) and 5-nitro-naphthalene-1-carboxylic acid (0.10 mmol). Purification by solid phase extraction (heptane/ethyl acetate=5:1) furnished 31.8 mg (90%) of the desired product as a solid (LC-MS: 97% UV-purity and 99% ELSD-purity). An analytical sample was obtained as slight yellow needles by recrystallization from diethylether (mp: 71-73° C.). ¹H-NMR δ3.36 (t, 2H, J=6.8), 4.60 (t, 2H, J=6.6), 7.22 (t, 1H, J=7.3), 7.31 (t, 2H, J=7.5), 7.46 (d, 2H, J=7.5), 7.65-7.71 (m, 2H), 8.18 (d, 1H, J=7.1), 8.19 (d, 1H, J=7.5), 8.66 (d, 1H, J=8.5), 9.26 (d, 1H, J=8.0).

Diethyl[2-(4-imidazol-1-yl-phenoxy)ethyl]amine (entry 5) was prepared from 2-diethylamino-ethanol (0.20 mmol) and 4-imidazol-1-yl-phenol (0.10 mmol). Purification by solid phase extraction (heptane/ethyl acetate=5:1) furnished 16.1 mg (62%) of the desired product as an oil (GC-MS: 100% purity). ¹H-NMR δ1.08 (t, 6H, J=7.1), 2.65 (q, 4H, J=7.2), 2.90 (t, 2H, J=6.1), 4.07 (t, 2H, J=6.1), 6.98 (d, 2H, J=9.0), 7.18 (s, 1H), 7.20 (s, 1H), 7.29 (m, 1H, J=8.5), 7.76 (s, 1H).

3-(4-Methoxy-phenoxy)-1-aza-bicyclo[2.2.2]octane (entry 6) was prepared from 1-aza-bicyclo[2.2.2]octan-3-ol (0.20 mmol) and 4-methoxy-phenol (0.10 mmol). Purification by solid phase extraction (heptane/ethyl acetate=5:1) furnished 16.2 mg (69%) of the desired product as an oil (LC-MS: 37% UV-purity and 87% ELSD-purity). ¹H-NMR δ1.39 (m, 1H), 1.54 (m, 1H), 1.73 (m, 1H), 2.01 (m, 1H), 2.12 (m, 1H), 2.77 (m, 1H), 2.87 (m, 3H), 2.99 (m, 1H), 3.25 (m, 1H), 3.76 (s, 3H), 4.27 (m, 1H), 6.81 (m, 4H).

TABLE 3
Results of Example 1 (Mitsunobu reaction)

| | | | | | | Purity |
|---|---|---|---|---|---|---|
| En- | | | | Yield | Purity | ELSD |
| try | Substrate 1 | Substrate 2 | Product | % | UV % | % |
| 1 | 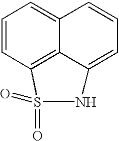 | 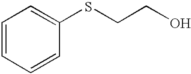 | 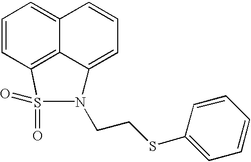 | 89 | 98 | 99 |
| 2 | 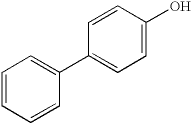 | 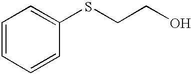 | 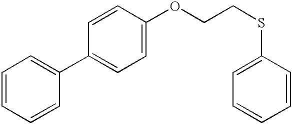 | 91 | 89 | 87 |
| 3 | 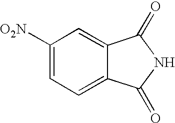 | 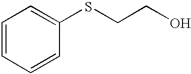 | 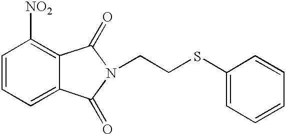 | 100 | 84 | 80 |
| 4 | 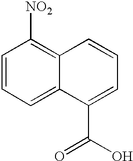 | 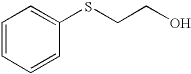 | 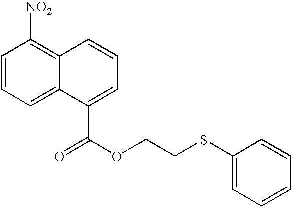 | 90 | 97 | 99 |
| 5 | 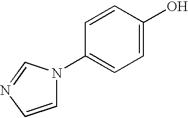 | 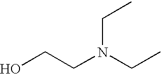 | 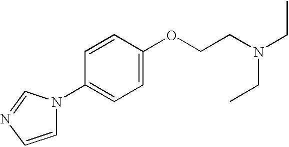 | 61 | 100 (GC- MS) | — |
| 6 | 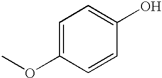 | 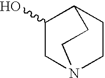 | 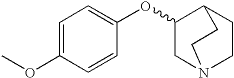 | 69 | 37 | 87 |

### Example 2

Acylation reaction by Use of Tablets Containing Polystyrene, Tetrabromomethane and Diphenylphosphanyl Polystyrene in Combination with Tablets of Isocyanato Methyl Polystyrene

Results of the evaluation are summarised in Table 4. A detailed description of the procedure follows:

4-Morpholinocarbonyl-ferrocene (entry 6). A tablet containing 0.11 mmol tetrabromomethane and 0.15 mmol resin bound diphenylphosphine was added at 0° C. to a solution of ferrocenecarboxylic acid (23.3 mg, 0.10 mmol), morpholine (10.7 mg, 0.12 mmol) and triethylamine (22.6 mg, 0.22 mmol) in dry THF (1.5 mL). After stirring for 16 h at room temperature, THF (2 mL) and a tablet (150 mg) comprising isocyanatomethyl polystyrene (0.15 mmol) were added. The mixture was stirred for 2 h at 60° C. The resin was filtered and washed with methylene chloride (1×1 mL), methanol (1×1 mL), and methylene chloride (1×2 mL). The solvents were evaporated in vacuo and the residue was purified by solid phase extraction (heptane/ethyl acetate=1:1) to furnish 13.6 mg (45%) as a orange/brown solid (LC-MS: 98% UV-purity and 99% ELSD-purity); ¹H-NMR δ3.69 (m, 4H), 3.74 (m, 4H), 4.24 (s, 5H), 4.32 (t, 2H, J=1.7), 4.55 (t, 2H, J=1.9).

N-(2,6-Dimethyl-phenyl)-4-niethoxy-benzainide (entry 1) was prepared from 4-methoxy benzoic acid (0.10 mmol) and 2,6-dimethyl aniline (0.10 mmol). Purification by solid phase extraction (heptane/ethyl acetate=1:1) furnished 7.4 mg (29%) of the desired product as a colourless solid (LC-MS: 66% UV-purity and 81% ELSD-purity). ¹H-NMR δ2.27 (s, 6H), 3.88 (s, 3H), 6.98 (d, 2H, J=7.5), 7.13 (m, 3H), 7.31 [s (broad), 1H], 7.89 (d, 2H, J=8.9). According to ¹H-NMR, the main impurity is 4-methoxy benzoic acid, which was used as starting material.

4-Chloro-N-(2-morpholin-4-yl-ethyl)-benzamide (entry 2) was prepared from 4-chloro benzoic acid (0.10 mmol) and 2-morpholin-4-yl-ethylamine (0.10 mmol). Purification by solid phase extraction (ethyl acetate/heptane/triethylamine=5:1:0.1) furnished 12.2 mg (45%) of the desired product as a colourless solid (LC-MS: 98% UV-purity and 99% ELSD-purity). ¹H-NMR δ2.51 [m (broad), 4H), 2.61 (t, 2H, J=5.9), 3.55 (q, 2H, J=5.7), 3.73 (t, 4H, J=4.7), 6.75 [s (broad), 1H], 7.42 (d, 2H, J=8.5), 7.71 (d, 2H, J=8.5).

Dodecanoic acid dipropylamide (entry 3) was prepared from dodecanoic acid (0.10 mmol) and dipropylamine (0.10 mmol). Purification by solid phase extraction (heptane/ethyl acetate=1:1) furnished 20.4 mg (72%) of the desired product as a colourless oil (LC-MS: 99% ELSD-purity). ¹H-NMR δ0.88 (t, 6H, J=7.3), 0.92 (t, 3H, J=7.5), 1.26 (m, 18H), 1.5-1.7 (m, 4H), 2.28 (t, 2H, J=7.8), 3.18 (t, 2H, J=7.8), 3.27 (t, 2H, J=7.8).

1-Morpholin-4-yl-4-phenyl-butan-1-one (entry 4) was prepared from 4-phenyl-butyric acid (0.10 mmol) and morpholine (0.10 mmol). Purification by solid phase extraction (heptane/ethyl acetate=1:1) furnished 17.4 mg (75%) of the desired product as a colourless oil (LC-MS: 65% UV-purity and 92% ELSD-purity). ¹H-NMR δ1.99 (qui, 2H, J=7.5), 2.31 (t, 2H, J=7.8), 2.68 (t, 2H, J=7.5), 3.37 (t, 2H, J=4.9), 3.5-3.7 (m, 6H), 7.19 (d, 2H, J=7.5), 7.21 (t, 1H, J=7.3), 7.29 (t, 2H, J=7.8).

Biphenyl-4-carboxylic acid pyridin-2-ylamide (entry 5) was prepared from 4-biphenylcarboxylic acid (0.10 mmol) and 2-aminopyridine (0.10 mmol). Purification by solid phase extraction (heptane/ethyl acetate=1:1) furnished 18.0 mg (66%) of the desired product as a slightly brown solid (LC-MS: 43% UV-purity and 72% ELSD-purity). ¹H-NMR δ7.14 (dd, 1H, J=7.1, J=5.2), 7.41 (t, 1H, J=6.8), 7.48 (t, 2H, J=8.0), 7.65 (d, 2H, J=8.0), 7.70 (d, 2H, J=8.0), 7.84 (t, 1H, J=7.8), 8.13 (d, 2H, J=8.0), 8.32 (d, 1H, J=3.8), 7.52 (d, 1H, J=8.5), 9.82 [s (broad), 1H]. According to ¹H-NMR, the main impurity is 4-biphenylcarboxylic acid, which was used as starting material.

TABLE 4
Results of Example 2 (Acylation Reaction)

| | | | | | | Purity |
|---|---|---|---|---|---|---|
| | | | | Yield | Purity | ELSD |
| Entry | Substrate 1 | Substrate 2 | Product | % | UV % | % |
| 1 | 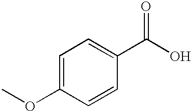 | 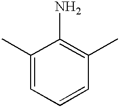 | 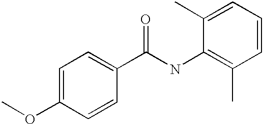 | 29 | 66 | 81 |
| 2 | 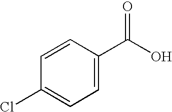 | 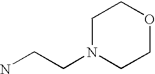 | 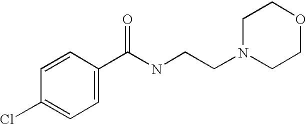 | 45 | 98 | 99 |
| 3 | 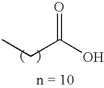 | 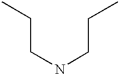 | 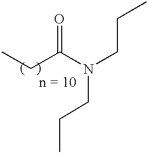 | 72 | — | 99 |
| 4 | 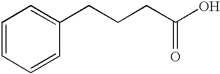 | 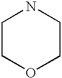 | 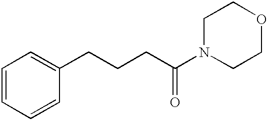 | 75 | 65 | 92 |
| 5 | 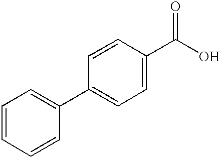 | 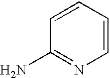 | 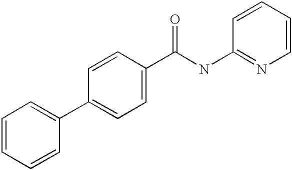 | 66 | 43 | 72 |
| 6 | 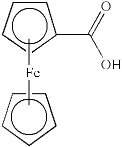 | 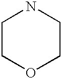 | 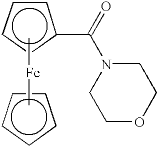 | 45 | 98 | 99 |

What is claimed is:

1. A dosing form for at least one solid reagent for use in synthetic or analytical chemistry that is compressed tablets each comprising essentially the same predetermined amount of said at least one reagent embedded in a polymer matrix comprising beads of a polymer insoluble in the solvent for the intended synthesis, and said tablets being capable of disintegrating in said solvent thereby dispersing the polymer beads and the at least one reagent into the solvent, wherein said tablets comprise less than 20 percent by weight of polyethylene glycol.

2. The dosing form according to claim 1, wherein the tablets are capable of disintegrating in the intended solvent within 10 minutes.

3. The dosing form according to claim 1, wherein the polymer matrix comprises a polymer selected among: polystyrene, a polymer having a backbone comprising styrene or substituted styrene monomers, copolymers comprising styrene or substituted styrene monomers, polystyrene crosslinked with divinyl benzene, polystyrene cross-linked with polyethylene glycol including the POEPS and POEPS-3 resins, polystyrene resins cross-linked with polyoxybutylene, polyethylene glycol grafted resins, polyoxyethylene polyoxy propylene resins and polymers co-polymerized together with magnetites or magnetites captured in highly cross-linked polystyrene particles.

4. The dosing form according to claim 1, wherein the polymer matrix further comprises an additive.

5. The dosing form according to claim 4, wherein the additive comprises a disintegrating agent.

6. The dosing form according to claim 5, wherein the disintegrating agent is polystyrene or dimethylated polyethyleneglycol having a molecular weight of about 2000 Da (DM-PEG 2000) or higher.

7. The dosing form according to claim 1, wherein the tablets are uncoated.

8. The dosing form according to claim 1, wherein the reagent is non soluble or almost non soluble in the solvent for which the tablets are intended.

9. The dosing form according to claim 1, wherein the solid reagent is a reagent that is useable in chemical synthesis.

10. The dosing form according to claim 9, wherein the solid reagent is selected among the following reagent types: Acetoxylating reagents, acid acceptors, acid catalysts, acrylating reagents, activated ester reagents, activating reagents, acyl anion equivalents, acylating reagents, acylation catalysts, aldolization reagents, alkene addition reagents, alkene metathesis catalysts, alkenylating reagents, alkenylation catalysts, alkoxide bases, alkylating reagents, alkylation catalysts, alkynylating reagents, allenylating reagents, allylating reagents, allylation catalysts, amide bases, amidine bases, aminating reagents, amination catalysts, amine bases, aminoalkylating reagents, aminomethylenating reagents, amphiphilic reagents, anion activation reagents, annulation reagents, arene alkylating reagents, arsenating reagents, arylating reagents, arylation catalysts, autoxidation catalysts, azide sources, bases, benzyne precursors, bis-annulating reagents, borylating reagents, bromination reagents, Bronsted-Lowry acids, carbamoylating reagents, carbene precursors, carboalumination reagents, carbon nucleophiles, carbonyl alkenation reagents, carbonylation reagents and catalysts, carboxamininylating reagents, carboxylation reagents, chelation reagents, chiral reagents, cleavage reagents, condensation catalysts, cross-coupling reagents, cuprating reagents, cyanating reagents, cyclization catalysts, cyclization reagents, cycloaddition catalysts, cycloaddition reagents, cyclopropanating reagents, dealkylating reagents, decarboxylating reagents, dehalogenating reagents, dehydrating reagents, dehydrogenating reagents, dehydrohalogenating reagents, deoxygenation reagents, deprotection reagents, derivatization reagents, desilylation reagents, desulfurization reagents, diazoalkane reagents, diazo transfer reagents, dihydroxylation reagents, elimination-inducing reagents, enolate equivalents, enophiles, epoxidizing reagents, ester hydrolis reagents, esterification reagents, fluorinating reagents, fluoroalkylating reagents, formylating reagents, glycosylation reagents, guanylating reagents, halogenating reagents, heteroatom nucleophiles, heterocyclic synthesis reagents, homoenolates, homologating reagents, hydration catalysts, hydride donors, hydroaluminating reagents, hydroborating reagents, hydrocyanation reagents, hydroformylation reagents, hydrogenation catalysts, hydrogen atom donors, hydrogenolysis catalysts, hydrohalogenating reagents, hydrosilylation catalysts and reagents, hydroxyalkylating reagents, hydroxymethylating reagents, isomerization catalysts, ketene precursors, Lewis acids and bases, metalating reagents, methoxylation reagents, methylation reagents, Michael acceptors, Michael addition catalysts, Michael donors, nitrating reagents, nitrosating reagents, nucleotide coupling reagents, oligomerization catalysts, oxidation catalysts, oxidative coupling reagents, oxidizing reagents, oxygenating reagents, peptide coupling reagents, phase-transfer catalysts, reagents, thiophilic reagents, transition metal ligands, trifluoromethylation reagents, vinylating reagents, vinylation catalysts, phenoxylating agents, phosphinylating reagents, phosphitylating reagents, phosphonylating reagents, phosphorylating reagents, photocycloaddition reagents, propargylating reagents, protecting reagents, radical promoters and reagents, rearrangement catalysts, rearrangement reagents, reducing reagents, resolving reagents, ring contraction reagents, ring expansion reagents, selenenylating and selenurating reagents, silylating reagents, stannylating reagents, sulfenylating reagents, sulfinylating reagents, sulfonylating reagents, sulfurating reagents, surfactants, tellurating reagents, thiocyanating reagents, thioetherification reagents, thionating reagents.

11. The dosing form according to claim 1, wherein the solid reagents are selected among: acetals, acids, alcohols and alkoxides, aldehydes, alkenes, alkynes, allenes, aluminum containing reagents, anions, antimony containing reagents, arsenic reagents, barium containing reagents, bases, biocatalysts, bismuth containing reagents, boron reagents, bromine containing reagents, cadmium containing reagents, calcium containing reagents, carboxylic derivatives, catalysts, cations, cerium containing reagents, cesium containing reagents, chiral reagents, chlorine reagents, chromium containing reagents, cobalt containing reagents, copper reagents, cyclopropanes, dienes and trienes, enzymes, erbium containing reagents, ethers, europium reagents, Fischer and Schrock carbene complexes, fluorine containing reagents, germanium containing reagents, gold containing reagents, hafnium containing reagents, halonium ions, heterocycles, hydrides, hydroxides, indium reagents, iodine containing reagents, iridium containing reagents, iron containing reagents, ketenes and ketene derivatives, ketones, lanthanide containing reagents, lanthanum containing reagents, lead containing reagents, lithium containing reagents, magnesium containing reagents, manganese containing reagents, mercury containing reagents, metal complexing agents, molybdenum containing reagents, nickel containing reagents, niobium containing reagents, nitrogen containing reagents, orthoesters, osmium containing reagents, oxonium ions, oxygen containing reagents, palladium containing reagents, peroxides, phenols, phosphorus reagents, platinum containing reagents, potassium containing reagents, quinones, rhenium containing reagents, rhodium containing reagents, ruthenium containing reagents, samarium containing reagents, selenium containing reagents, silicon containing reagents, silver containing reagents, sodium reagents, sulfur reagents, tantalum containing reagents, tellurium containing reagents, thallium containing reagents, tin containing reagents, titanium containing reagents, tungsten containing reagents, uranium containing reagents, vanadium containing reagents, xenon containing reagents, ylides, ytterbium containing reagents, zinc containing reagents, zirconium reagents.

12. The dosing form according to claim 1, wherein at least one reagent is bound to the polymer.

13. The dosing form according to claim 1, wherein the reagents comprise a phosphine and an azo compound of the formula 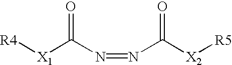
wherein X₁ and X₂ independently are N or O, and R⁴ and R⁵ independently are selected from the group comprising lower alkyl and polymer-bonded equivalents thereof.

14. The dosing form according to claim 13, wherein the phosphine or the azo compound is bonded to the polymer.

15. The dosing form according to claim 13, wherein the phosphine is of the formula R¹R²R³P wherein R¹, R² and R³ independently are selected from the group comprising phenyl, heteroaryl, lower alkyl, phenyl-lower alkyl, heteroaryl-lower aryl and polymer-bonded equivalents thereof.

16. The dosing form according to claim 1, wherein the reagents comprise a phosphine and carbon tetrabromide.

17. The dosing form according to claim 16, wherein the phosphine is bonded to the polymer.

18. The dosing form according to claim 16, wherein the phosphine is of the formula R¹R²R³P wherein R¹, R² and R³ independently are selected from the group comprising phenyl, heteroaryl, lower alkyl, phenyl-lower alkyl, heteroaryl-lower aryl and polymer-bonded equivalents thereof.

19. A method of alkylation of acidic heteroatoms using a dosing form according to claim 13.

20. A method of acylation of basic heteroatoms using a dosing form according to claim 16.

21. A method of using a dosing form according to claim 1 in synthetic or analytical chemistry.

22. A method of carrying out parallel synthesis, split and mix synthesis, or combinatorial synthesis using a dosing form according to claim 1.

23. A method for production of a dosing form for at least one solid reagent for use in synthetic or analytical chemistry comprising compression of polymer beads mixed with said at least one reagent and optional additives into tablets each comprising essentially the same predetermined amount of said at least one reagent embedded in a polymer matrix comprising beads of a polymer insoluble in the solvent for the intended synthesis, and said tablets being capable of disintegrating in said solvent thereby dispersing the polymer beads and the at least one reagent into the solvent, wherein the polymer or a mixture of the polymer and at least one reagent is pre-treated with an aprotic organic solvent and dried before tablet formation.

24. The method according to claim 23, wherein the pre-treatment comprises suspending the polymer or a mixture of the polymer and at least one reagent in an aprotic organic solvent and subsequently drying said polymer or mixture of polymer and reagent before tablet formation.

25. The method according to claim 23, wherein the pre-treatment comprises suspending the polymer or a mixture of the polymer and at least one reagent in an aprotic organic solvent wherein a reagent soluble in said solvent is dissolved, and subsequently depositing said dissolved reagent with said polymer or polymer reagent mixture by evaporation of the solvent before tablet formation.

26. The method according to claim 23, wherein the aprotic organic solvent is methylene chloride or tetrahydrofuran.

27. The method according to claim 23, wherein the material used for tabletting comprises less than 20 percent by weight of polyethylene glycol.

28. The method according to claim 23, wherein the manufactured tablets are capable of disintegrating in the intended solvent within 10 minutes.

29. The method claim 23, wherein the polymer matrix comprises a polymer selected among: polystyrene, a polymer having a backbone comprising styrene or substituted styrene monomers, copolymers comprising styrene or substituted styrene monomers, polystyrene crosslinked with divinyl benzene, polystyrene cross-linked with polyethylene glycol including the POEPS and POEPS-3 resins, polystyrene resins cross-linked with polyoxybutylene, polyethylene glycol grafted resins, polyoxyethylene polyoxy propylene resins and polymers co-polymerized together with magnetites or magnetites captured in highly cross-linked polystyrene particles.

30. The method according claim 23, wherein the material used for tabletting further comprises an additive.

31. The method according to claim 30, wherein the additive comprises a disintegrating agent.

32. The method according to claim 31, wherein the disintegrating agent is polystyrene or dimethylated polyethyleneglycol having a molecular weight of about 2000 Da (DM-PEG 2000) or higher.

33. The method according to claim 23, wherein the reagent is non soluble or almost non soluble in the solvent for which the tablets are intended.

34. The method according to claim 23, wherein the solid reagent is a reagent that is useable in chemical synthesis.

35. The method according to claim 23, wherein the solid reactant is selected among the following reagent types: Acetoxylating reagents, acid acceptors, acid catalysts, acrylating reagents, activated ester reagents, activating reagents, acyl anion equivalents, acylating reagents, acylation catalysts, aldolization reagents, alkene addition reagents, alkene metathesis catalysts, alkenylating reagents, alkenylation catalysts, alkoxide bases, alkylating reagents, alkylation catalysts, alkynylating reagents, allenylating reagents, allylating reagents, allylation catalysts, amide bases, amidine bases, aminating reagents, amination catalysts, amine bases, aminoalkylating reagents, aminomethylenating reagents, amphiphilic reagents, anion activation reagents, annulation reagents, arene alkylating reagents, arsenating reagents, arylating reagents, arylation catalysts, autoxidation catalysts, azide sources, bases, benzyne precursors, bis-annulating reagents, borylating reagents, bromination reagents, Brønsted-Lowry acids, carbamoylating reagents, carbene precursors, carboalumination reagents, carbon nucleophiles, carbonyl alkenation reagents, carbonylation reagents and catalysts, carboxamininylating reagents, carboxylation reagents, chelation reagents, chiral reagents, cleavage reagents, condensation catalysts, cross-coupling reagents, cuprating reagents, cyanating reagents, cyclization catalysts, cyclization reagents, cycloaddition catalysts, cycloaddition reagents, cyclopropanating reagents, dealkylating reagents, decarboxylating reagents, dehalogenating reagents, dehydrating reagents, dehydrogenating reagents, dehydrohalogenating reagents, deoxygenation reagents, deprotection reagents, derivatization reagents, desilylation reagents, desulfurization reagents, diazoalkane reagents, diazo transfer reagents, dihydroxylation reagents, elimination-inducing reagents, enolate equivalents, enophiles, epoxidizing reagents, ester hydrolis reagents, esterification reagents, fluorinating reagents, fluoroalkylating reagents, formylating reagents, glycosylation reagents, guanylating reagents, halogenating reagents, heteroatom nucleophiles, heterocyclic synthesis reagents, homoenolates, homologating reagents, hydration catalysts, hydride donors, hydroaluminating reagents, hydroborating reagents, hydrocyanation reagents, hydroformylation reagents, hydrogenation catalysts, hydrogen atom donors, hydrogenolysis catalysts, hydrohalogenating reagents, hydrosilylation catalysts and reagents, hydroxyalkylating reagents, hydroxymethylating reagents, isomerization catalysts, ketene precursors, Lewis acids and bases, metalating reagents, methoxylation reagents, methylation reagents, Michael acceptors, Michael addition catalysts, Michael donors, nitrating reagents, nitrosating reagents, nucleotide coupling reagents, oligomerization catalysts, oxidation catalysts, oxidative coupling reagents, oxidizing reagents, oxygenating reagents, peptide coupling reagents, phase-transfer catalysts, reagents, thiophilic reagents, transition metal ligands, trifluorometbylation reagents, vinylating reagents, vinylation catalysts, phenoxylating agents, phosphinylating reagents, phosphitylating reagents, phosphonylating reagents, phosphorylating reagents, photocycloaddition reagents, propargylating reagents, protecting reagents, radical promoters and reagents, rearrangement catalysts, rearrangement reagents, reducing reagents, resolving reagents, ring contraction reagents, ring expansion reagents, selenenylating and selenurating reagents, silylating reagents, stannylating reagents, sulfenylating reagents, sulfinylating reagents, sulfonylating reagents, sulfurating reagents, surfactants, tellurating reagents, thiocyanating reagents, thioetherification reagents, thionating reagents.

36. The method according to claim 23, wherein the solid reactant is selected among: acetals, acids, alcohols and alkoxides, aldehydes, alkenes, alkynes, allenes, aluminum containing reagents, anions, antimony containing reagents, arsenic reagents, barium containing reagents, bases, biocatalysts, bismuth containing reagents, boron reagents, bromine containing reagents, cadmium containing reagents, calcium containing reagents, carboxylic derivatives, catalysts, cations, cerium containing reagents, cesium containing reagents, chiral reagents, chlorine reagents, chromium containing reagents, cobalt containing reagents, copper reagents, cyclopropanes, dienes and trienes, enzymes, erbium containing reagents, ethers, europium reagents, Fischer and Schrock carbene complexes, fluorine containing reagents, germanium containing reagents, gold containing reagents, hafnium containing reagents, halonium ions, heterocycles, hydrides, hydroxides, indium reagents, iodine containing reagents, iridium containing reagents, iron containing reagents, ketenes and ketene derivatives, ketones, lanthanide containing reagents, lanthanum containing reagents, lead containing reagents, lithium containing reagents, magnesium containing reagents, manganese containing reagents, mercury containing reagents, metal complexing agents, molybdenum containing reagents, nickel containing reagents, niobium containing reagents, nitrogen containing reagents, orthoesters, osmium containing reagents, oxonium ions, oxygen containing reagents, palladium containing reagents, peroxides, phenols, phosphorus reagents, platinum containing reagents, potassium containing reagents, quinones, rhenium containing reagents, rhodium containing reagents, ruthenium containing reagents, samarium containing reagents, selenium containing reagents, silicon containing reagents, silver containing reagents, sodium reagents, sulfur reagents, tantalum containing reagents, tellurium containing reagents, thallium containing reagents, tin containing reagents, titanium containing reagents, tungsten containing reagents, uranium containing reagents, vanadium containing reagents, xenon containing reagents, ylides, ytterbium containing reagents, zinc containing reagents, zirconium reagents.

37. The method according claim 23, wherein at least one reagent is bound to the polymer.

38. A dosing form for at least one solid reagent for use in synthetic or analytical chemistry being compressed tablets each comprising essentially the same predetermined amount of said at least one reagent embedded in a polymer matrix comprising beads of a polymer insoluble in the solvent for the intended synthesis, and said tablets being capable of disintegrating in said solvent thereby releasing the at least one reagent and dispersing the matrix as polymer beads into the solvent, wherein said tablets are made by a method according to claim 23.

39. A method of using a dosing form according to claim 38 in synthetic or analytical chemistry.

40. A method of carrying out parallel synthesis, split and mix synthesis or combinatorial chemistry using a dosing form according to claim 38.
